(19) United States
(12) Patent Application Publication
(10) Pub. No.: US 2011/0245685 A1
(43) Pub. Date: Oct. 6, 2011

(54) BLOOD VESSEL DISPLAY DEVICE

(75) Inventors: Akihiro MURATA, Hokuto (JP); Takashi TAKEDA, Suwa (JP)

(73) Assignee: SEIKO EPSON CORPORATION, Tokyo (JP)

(21) Appl. No.: 13/078,049

(22) Filed: Apr. 1, 2011

(30) Foreign Application Priority Data
Apr. 2, 2010 (JP) 2010-085834

(51) Int. Cl. A61B 6/00

(52) U.S. Cl. 600/476

(57) ABSTRACT

A blood vessel display device includes: a detection unit that scans body tissue with laser light for detection and detects reflected light from an irradiated part of the body tissue to which the laser light for detection has been irradiated; an image data generating unit that detects a surface shape of the irradiated part and the arrangement of blood vessels, which are present in a superficial layer of the irradiated part, on the basis of a detection result of the detection unit and generates image data of an image for visualizing the blood vessels displayed on the irradiated part; and a display unit that displays an image for visualizing the blood vessels on the irradiated part by scanning the irradiated part with laser light for display on the basis of the image data generated by the image data generating unit.

### BACKGROUND

1. Technical Field

The present invention relates to a blood vessel display device.

2. Related Art

In the medical field, for example, when a doctor or the like gives a patient an injection in his or her arm, the doctor or the like finds a blood vessel in the superficial layer and inserts an injection needle beneath the skin tissue. In this case, if a doctor can visually identify a blood vessel, the probability that the blood vessel will be obtained is high. However, there are some patients whose blood vessels are difficult to identify visually, for example, a child or a person with thick subcutaneous fat. In such a case, it is very difficult to obtain the blood vessels.

In recent years, therefore, a device (“VEINVIEWER” made by Luminetx) which visualizes blood vessels by displaying blood vessel image data, which is acquired by a known vein identification technology, in real time on the body tissue surface (for example, the back of a hand) using a DLP projector or the like has been under development as a technique for visualizing blood vessels present in the superficial layer of body tissue (for example, refer to “Luminetx VEINVIEWER [online] 2006 [accessed on Mar. 5, 2010], Internet <URL: http://www.luminetx.com/Portals/0/pdf/VVGS%20General%20%20 Broch%20(D00144F).pdf>”).

In such a device, however, it is necessary to perform acquisition of the blood vessel image data and display of the blood vessel image data by using separate devices. For this reason, an error occurs between the position of a blood vessel visualized by a blood vessel image and the position of an actual blood vessel. In addition, the body tissue surface is not flat but complexly curved. For this reason, when an image is displayed on the body tissue surface using a DLP projector or the like, a problem also occurs in which defocusing occurs and an image is blurred accordingly. That is, a device in the related art has a problem in that it is not possible to display a blood vessel image, which is clear and has no positional deviation from the actual blood vessels, on the body tissue surface.

### SUMMARY

An advantage of some aspects of the invention is to provide a blood vessel display device capable of displaying a blood vessel image, which is clear and has no positional deviation from the actual blood vessels, on the body tissue surface.

According to an aspect of the invention, there is provided a blood vessel display device including: a detection unit that scans body tissue with laser light for detection and detects reflected light from the irradiated part of the body tissue to which the laser light for detection has been irradiated; an image data generating unit that detects the surface shape of the irradiated part and the arrangement of blood vessels, which are present in a superficial layer of the irradiated part, on the basis of a detection result of the detection unit and generates image data of an image for visualizing the blood vessels displayed on the irradiated part; and a display unit that displays an image for visualizing the blood vessels on the irradiated part by scanning the irradiated part with laser light for display on the basis of the image data generated by the image data generating unit.

In this case, a blood vessel image (image which visualizes blood vessels) which is clear and has no positional deviation from the actual blood vessels can be displayed on the body tissue surface. In addition, since laser light is used as display light, a blood vessel image which is clear and has no blur can be displayed even if the body tissue surface is complexly curved.

In the blood vessel display device according to the aspect of the invention, it is preferable to further include a control unit that controls driving of the detection unit and also controls driving of the display unit on the basis of the image data generated by the image data generating unit.

In this case, a blood vessel image can be more reliably displayed on the body tissue surface.

In the blood vessel display device according to the aspect of the invention, it is preferable that the detection unit includes a detection laser light source which emits the laser light for detection, a detection laser light scanning section which scans the body tissue with the laser light for detection emitted from the detection laser light source, and a light receiving element which receives the reflected light and the display unit includes a display laser light source which emits the laser light for display and a display laser light scanning section which scans the irradiated part with the laser light for display emitted from the display laser light source.

In this case, the device configuration of the blood vessel display device becomes simple.

In the blood vessel display device according to the aspect of the invention, it is preferable that each of the detection laser light scanning section and the display laser light scanning section includes an actuator which is provided such that a movable plate including a light reflecting section with light reflectivity is rotatable in at least one direction and which scans the irradiated part with laser light reflected from the light reflecting section due to the rotation.

In this case, the device configuration of the light scanning section becomes simple, and excellent laser light scanning characteristics can be realized.

In the blood vessel display device according to the aspect of the invention, it is preferable that the detection laser light scanning section also serves as the display laser light scanning section.

In this case, the device configuration of the blood vessel display device becomes simple. In addition, since the laser light for display and the laser light for detection scan the irradiated part using the same light scanning section, a blood vessel image positioned correctly without deviation can be depicted on the irradiated part scanned by the laser light for detection.

In the blood vessel display device according to the aspect of the invention, it is preferable that the laser light for detection is near-infrared laser light.

Near-infrared laser light has a characteristic of being absorbed by hemoglobin contained in the blood flowing through the blood vessel. Accordingly, using such a characteristic, it is possible to more reliably detect a blood vessel present in the superficial layer of the irradiated part.

In the blood vessel display device according to the aspect of the invention, it is preferable that the image which visualizes the blood vessels is displayed by a green laser light for display.

In this case, an image in which blood vessels are more visualized can be displayed on the irradiated part.

In the blood vessel display device according to the aspect of the invention, it is preferable that the display unit visualizes the blood vessels and displays of an image of a target part of the blood vessels.

In this case, since a target part can be easily checked, it becomes easier to perform various medical treatments.

In the blood vessel display device according to the aspect of the invention, it is preferable that the target part is a part into which an injection needle is inserted.

In this case, it becomes easier to obtain a blood vessel when giving an injection (when inserting an injection needle beneath the skin).

In the blood vessel display device according to the aspect of the invention, it is preferable that the image data generating unit generates the image data at predetermined intervals.

In this case, even if the body tissue is displaced with respect to the blood vessel display device, it becomes possible to depict a new blood vessel image so as to follow the displacement. Therefore, a blood vessel image with no positional deviation from the actual blood vessels can be continuously displayed.

### BRIEF DESCRIPTION OF THE DRAWINGS

The invention will be described with reference to the accompanying drawings, wherein like numbers reference like elements.

FIG. 1 is a view showing an image displayed by a blood vessel display device according to an embodiment of the invention.

FIG. 2 is a schematic view showing the blood vessel display device according to the embodiment of the invention.

FIG. 3 is a perspective view showing the partial cross section of an optical scanner provided in the blood vessel display device shown in FIG. 2.

FIGS. 4A and 4B are cross-sectional views for explaining the driving of the optical scanner shown in FIG. 3.

FIG. 5 is a schematic view showing a blood vessel display device according to a second embodiment of the invention.

FIGS. 6A to 6C are views showing an example of an image displayed by a display unit shown in FIG. 5.

### DESCRIPTION OF EXEMPLARY EMBODIMENTS

Hereinafter, a blood vessel display device according to preferred embodiments of the invention will be described with reference to the accompanying drawings.

### First Embodiment

First, a blood vessel display device according to a first embodiment of the invention will be described.

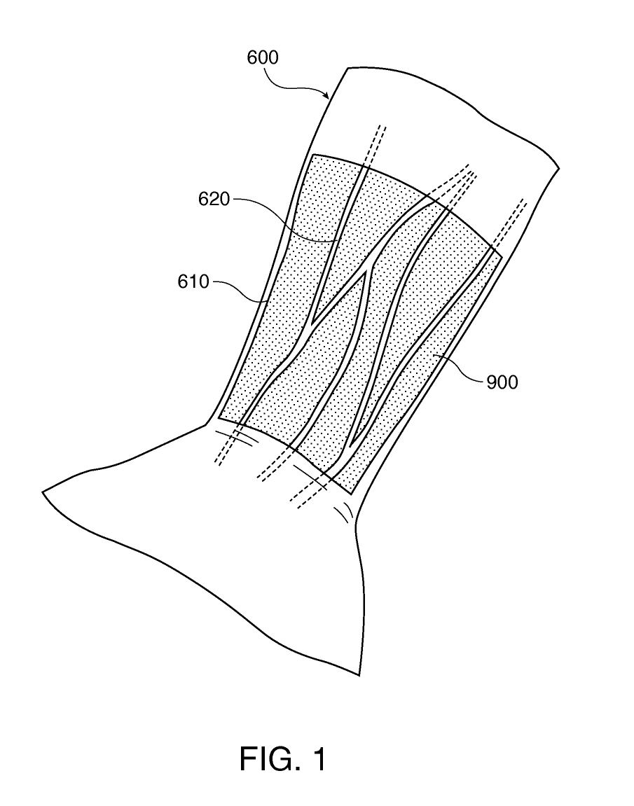

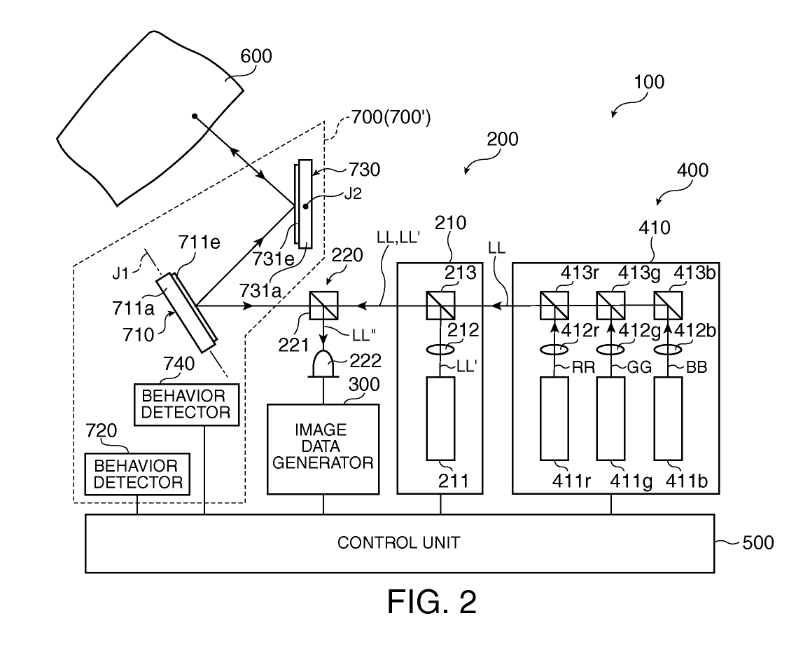

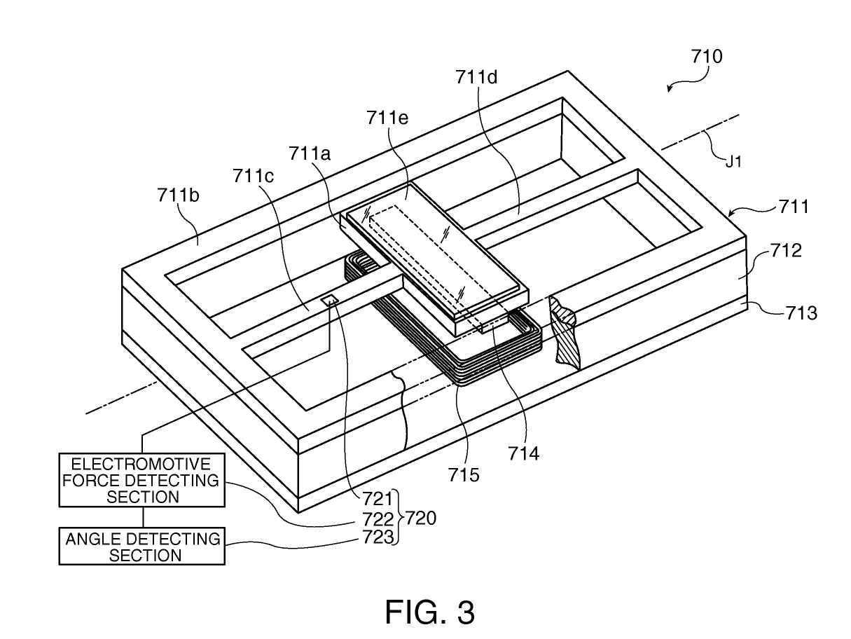

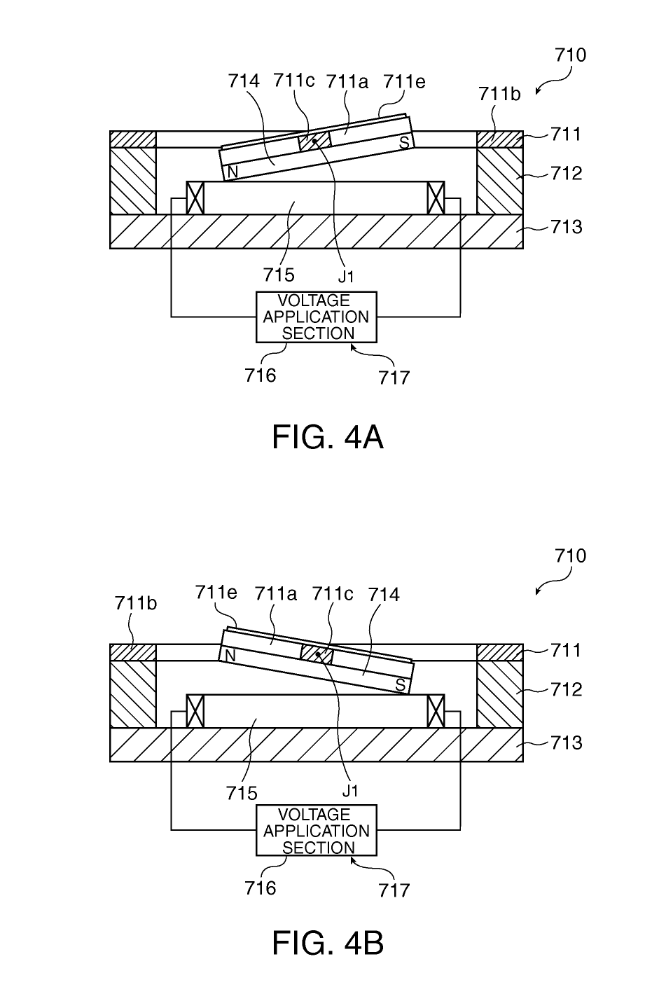

FIG. 1 is a view showing an image displayed by the blood vessel display device according to the first embodiment of the invention. FIG. 2 is a schematic view showing the blood vessel display device according to the first embodiment of the invention. FIG. 3 is a perspective view showing the partial cross section of an optical scanner provided in the blood vessel display device shown in FIG. 2. FIGS. 4A and 4B are cross-sectional views for explaining the driving of the optical scanner shown in FIG. 3. Moreover, in the following explanation, an upper side, a lower side, a left side, and a right side in FIGS. 3, 4A, and 4B are called “top”, “bottom”, “left”, and “right”, respectively, for the sake of convenience.

A blood vessel display device 100 is a device which visualizes a blood vessel (especially a vein) present in a superficial layer of body tissue. Such a blood vessel display device 100 is used to obtain a blood vessel reliably by visualizing a blood vessel 620 present in a superficial layer of a patient's arm 600 when a doctor or the like gives the patient an injection, for example, as shown in FIG. 1.

There are some patients whose blood vessels are difficult to identify visually, for example, like a child or a person with thick subcutaneous fat. If the blood vessel display device 100 is used, blood vessels which cannot be visually identified can be visualized even for such patients. Accordingly, blood vessels can be reliably obtained. As a result, a doctor can provide medical treatment to any kind of patient quickly, reliably, and safely. In addition, since erroneous insertion of an injection needle is prevented, the burden on a patient is also reduced.

Hereinafter, the blood vessel display device 100 will be described in detail.

As shown in FIG. 2, the blood vessel display device 100 includes a detector 200, an image data generator 300, a display unit 400, and a control unit 500. These constituent components will now be described one by one. Moreover, in the following explanation, as shown in FIG. 1, the case of visualizing the blood vessel 620 present in the superficial layer of the patient's arm 600 will be given as a representative example for the sake of convenience.

### Detector 200

The detector 200 has a function of scanning the patient's arm 600 with laser light for detection LL′ and detecting reflected laser light LL″ from an irradiated part 610 to which the laser light for detection LL′ has been irradiated.

As shown in FIG. 2, the detector 200 includes a detection laser light emitting device 210 which emits the laser light for detection LL′, a detection laser light scanning section 700 which scans the arm 600 with the laser light for detection LL′ emitted from the detection laser light emitting device 210, and a light receiving section 220 which receives the reflected laser light LL″ from the arm 600 (irradiated part 610). By adopting such a configuration, the configuration of the detector 200 becomes simple.

The detection laser light emitting device 210 includes a laser light source 211 and a collimator lens 212 and a dichroic mirror 213 which are provided corresponding to the laser light source 211. The laser light source 211 emits the laser light for detection LL′ according to a driving signal transmitted from the control unit 500. The emitted laser light for detection LL′ is collimated by the collimator lens 212 to become a narrow beam. The laser light for detection LL′ collimated by the collimator lens 212 is reflected by the dichroic mirror 213 and reaches the detection laser light scanning section 700.

Although the laser light for detection LL′ emitted from the laser light source 211 is not particularly limited, it is preferable that the laser light for detection LL′ is near-infrared laser light, specifically, laser light with a wavelength of about 600 nm to 900 nm. It is known that laser light with such a wavelength is absorbed by hemoglobin (erythrocyte) contained in blood flowing through the blood vessel. Accordingly, by using near-infrared laser light for detection LL′, it is possible to detect the blood vessel 620 present in the superficial layer of the irradiated part 610 more reliably and accurately, as will be described later.

The detection laser light scanning section 700 scans the superficial layer of the arm 600 in a two-dimensional manner with the laser light for detection LL′ emitted from the detection laser light emitting device.

As shown in FIG. 2, the detection laser light scanning section 700 includes: a first optical scanner 710 which scans the arm 600 with the laser light for detection LL′ emitted from the detection laser light emitting device 210 in a first direction with respect to the arm 600; a behavior detector 720 which detects the behavior of a movable plate 711a, which will be described later, provided in the first optical scanner 710; a second optical scanner 730 which scans the arm 600 with the laser light for detection LL′ in a second direction with respect to the arm 600 which is perpendicular to the first direction; and a behavior detector 740 which detects the behavior of a movable plate 731a, which will be described later, provided in the second optical scanner 730. By adopting such a configuration for the detection laser light scanning section 700, the device configuration of the detection laser light scanning section 700 can be simplified and excellent scanning characteristics can be realized for laser light (laser light for detection LL′ and laser light LL for display).

Hereinafter, for the sake of convenience, the configurations of the first and second optical scanners 710 and 730 will be specifically described. Since the first and second optical scanners 710 and 730 have the same configuration, only the first optical scanner 710 will be representatively described and an explanation regarding the second optical scanner 730 will be omitted.

As shown in FIG. 3, the first optical scanner 710 is a so-called one degree-of-freedom vibration system, and has abase 711, a counter substrate 713 provided to face the bottom surface of a base 711 and a spacer 712 provided between the base 711 and the counter substrate 713.

The base 711 has the movable plate 711a, a supporting section 711b which supports the movable plate 711a to be rotatable, and a pair of connecting sections 711c and 711d which connect the movable plate 711a to the supporting section 711b.

The movable plate 711a has an approximately rectangular shape in plan view. A light reflecting section (mirror) 711e with light reflectivity is provided on the movable plate 711a. For example, the light reflecting section 711e is formed of a metal film, such as Al or Ni. In addition, a permanent magnet 714 is provided below the movable plate 711a.

The supporting section 711b is provided to surround the outer periphery of the movable plate 711a in a plan view of the movable plate 711a. That is, the supporting section 711b has a frame shape, and the movable plate 711a is located thereinside.

The connecting section 711c connects the movable plate 711a to the supporting section 711b at one side of the movable plate 711a, and the connecting section 711d connects the movable plate 711a to the supporting section 711b at the other side of the movable plate 711a. Each of the connecting sections 711c and 711d has a longitudinal shape and may be elastically deformed. Such a pair of connecting sections 711c and 711d are coaxially provided, and the movable plate 711a rotates with respect to the supporting section 711b with the axis (hereinafter, referred to as a “rotation center axis J1”) as the center.

The base 711 is formed using silicon as a main material, for example. The movable plate 711a, the supporting section 711b, and the connecting sections 711c and 711d are integrally formed.

The spacer 712 has a frame shape, and the top surface of the spacer 712 is bonded to the bottom surface of the base 711. In addition, the shape of the spacer 712 is almost equal to that of the supporting section 711b in plan view of the movable plate 711a. The spacer 712 is formed of various kinds of glass or ceramics, silicon, or SiO₂, for example.

In addition, the method of bonding the spacer 712 to the base 711 is not particularly limited. For example, they may be bonded to each other with another member, such as an adhesive, interposed therebetween, or anodic bonding or the like may be used depending on a constituent material of the spacer 712.

Similar to the spacer 712, the counter substrate 713 is formed of various kinds of glass, silicon, or SiO₂, for example. A coil 715 is provided on the counter substrate 713 so as to face the movable plate 711a.

The permanent magnet 714 has a plate bar shape and is provided along the bottom surface of the movable plate 711a. The permanent magnet 714 is magnetized in a direction perpendicular to the rotation center axis J1 in plan view of the movable plate 711a. That is, the permanent magnet 714 is provided such that a line which connects the two poles (N and S poles) to each other is perpendicular to the rotation center axis J1.

Although the permanent magnet 714 is not particularly limited, it is possible to use a neodymium magnet, a ferrite magnet, a samarium cobalt magnet, and an alnico magnet, for example.

The coil 715 is provided so as to surround the outer periphery of the permanent magnet 714 in a plan view of the movable plate 711a.

Moreover, as shown in FIGS. 4A and 4B, the first optical scanner 710 has a voltage application section 716 which applies a voltage to the coil 715. The voltage application section 716 is configured to be able to adjust (change) various conditions, such as a voltage value or a voltage frequency to be applied. The voltage application section 716, the coil 715, and the permanent magnet 714 form a driving section 717 which rotates the movable plate 711a.

A predetermined voltage is applied from the voltage application section 716 to the coil 715 by control of the control unit 500, such that a predetermined current flows.

For example, if an AC voltage is applied from the voltage application section 716 to the coil 715 by control of the control unit 500, a current flows according to the voltage application. Then, a magnetic field is generated in a thickness direction of the movable plate 711a and the direction of the magnetic field is periodically changed. That is, a state where the vicinity of the top side of the coil 715 is an S pole and the vicinity of the bottom side of the coil 715 is an N pole and a state where the vicinity of the top side of the coil 715 is an N pole and the vicinity of the bottom side of the coil 715 is an S pole are alternately changed. As a result, the movable plate 711a rotates around the rotation center axis J1 while deforming the connecting sections 711c and 711d torsionally (states shown in FIGS. 4A and 4B are alternately repeated).

In addition, by adjusting the voltage applied from the voltage application section 716 to the coil 715 by control of the control unit 500, the flowing current can be adjusted. Accordingly, a deflection angle (amplitude) of the movable plate 711a around the rotation center axis J1 can be adjusted.

In addition, the configuration of such a first optical scanner 710 is not particularly limited so long as it is possible to rotate the movable plate 711a. For example, regarding the driving method, piezoelectric driving using a piezoelectric element or electrostatic driving using electrostatic attraction may be applied instead of electromagnetic driving using the coil 715 and the permanent magnet 714.

As shown in FIG. 2, the first optical scanner 710 with the above-described configuration and the second optical scanner 730 with the same configuration as the first optical scanner 710 are provided such that the rotation center axes J1 and J2 are perpendicular to each other. By providing the first and second optical scanners 710 and 730 in this way, the laser light for detection LL′ emitted from the detection laser light emitting device 210 can scan the surface of the arm 600 in a two-dimensional manner (in two directions perpendicular to each other).

Although the rotation speeds of the first and second optical scanners 710 and 730 are not particularly limited, it is preferable that the rotation speed of one optical scanner is faster than that of the other optical scanner. In this case, a scan characteristic of laser light (laser light for detection LL′ and laser light LL for display) which scans the arm 600 is improved. For example, when the rotation speed of the first optical scanner 710 is set to be faster than that of the second optical scanner 730, it is preferable to set resonance driving for the first optical scanner 710 and non-resonance driving for the second optical scanner 730. In this case, the effect described above becomes more noticeable.

Next, the behavior detector 720 which detects the behavior (angle) of the movable plate 711a of the first optical scanner 710 will be described. In addition, since the behavior detector 740 which detects the behavior (angle) of the movable plate 731a of the second optical scanner 730 has the same configuration as the behavior detector 720, the explanation will be omitted.

As shown in FIG. 3, the behavior detector 720 includes a piezoelectric element 721 provided on the connecting section 711c of the first optical scanner 710, an electromotive force detecting section 722 which detects an electromotive force generated from the piezoelectric element 721, and an angle detecting section 723 which detects the angle (deflection angle) of the movable plate 711a on the basis of a detection result of the electromotive force detecting section 722.

The piezoelectric element 721 deforms according to torsional deformation of the connecting section 711c caused by rotation of the movable plate 711a. The piezoelectric element 721 has a characteristic of generating an electromotive force corresponding to the amount of deformation when the piezoelectric element 721 deforms from the natural state where the external force is not given. Therefore, the angle detecting section 723 calculates the degree of torsion of the connecting section 711c on the basis of the size of the electromotive force detected by the electromotive force detecting section 722 and calculates the angle of the movable plate 711a from the degree of torsion. In this way, the behavior of the movable plate 711a is detected. The behavior of the detected movable plate 711a is transmitted from the angle detecting section 723 to the control unit 500.

In addition, detection of the behavior of the movable plate 711a may be performed in real time (continuously) or may be intermittently performed every predetermined time. In addition, the behavior detector 720 is not limited to the configuration using a piezoelectric element like the present embodiment if the behavior detector 720 can detect the behavior of the movable plate 711a. For example, the behavior of the movable plate 711a may be detected by providing a light receiving element, such as a photodiode, and a device, which emits laser light toward the light receiving element, such that laser light receiving of the light receiving element is blocked when the movable plate 711a is at a predetermined position and then detecting the timing at which laser light is blocked.

The laser light for detection LL′ (laser light for detection LL′ emitted at a predetermined time), which is emitted from the detection laser light scanning section 700 with the above-described configuration in order to scan the surface of the arm 600, is reflected from the surface (superficial layer) of the arm 600 to become the reflected laser light LL″ and reach the detection laser light scanning section 700 again. In this case, the reflected laser light LL″ will return to the detection laser light emitting device 210 through the same optical path as that which it follows to scan the surface of the arm 600. The light receiving section 220 branches the reflected laser light LL″ trying to return to the detection laser light emitting device 210 on the way and receives it.

In addition, not only the reflected laser light LL″ but also reflected light from parts other than the part to which the laser light for detection LL′ is irradiated may enter the detection laser light scanning section 700. However, since the incident angle of the reflected light with respect to a light reflecting section 731e of the second optical scanner 730 is different from that of the reflected laser light LL″, the reflected light is not received by a photodiode 222 unlike the reflected laser light LL″. From such a point, the photodiode 222 can receive only the reflected laser light LL″ reliably.

As shown in FIG. 2, the light receiving section 220 includes a beam splitter 221, which is provided to overlap the optical path of the laser light for detection LL′ until it reaches the detection laser light scanning section 700 after emission from the detection laser light emitting device 210 and which branches the reflected laser light LL″, and the photodiode (light receiving element) 222 which receives the reflected laser light LL″ branched by the beam splitter 221.

Image Data Generator 300

The image data generator 300 has a function of detecting the surface shape of the irradiated part 610 and the arrangement of the blood vessel 620 present in a superficial layer of the irradiated part 610 on the basis of a detection result of the detector 200, that is, on the basis of the reflected laser light LL″ received by the photodiode 222 and of generating image data 900D of an image 900 for visualizing the blood vessel which is displayed on the irradiated part 610.

Such an image data generator 300 generates the image data 900D as follows, for example. In addition, the method of generating the image data 900D is not limited to the following method. For example, although the method described below is a method using a TOF (Time Of Flight) method, it may be a method using a phase difference detecting method or trigonometry instead of this.

First, driving of the first and second optical scanners 710 and 730 is started by the control unit 500. Then, the control unit 500 controls the detection laser light emitting device 210 to emit pulsed laser light for detection LL′ (hereinafter, referred to as laser light for detection LL1′) at a predetermined timing. In this case, the control unit 500 stores an emission time and a scanning direction (in other words, postures of the movable plates 711a and 731a) of the laser light for detection LL1′ so as to match each other on the basis of a clock signal, behavior signals of the first and second optical scanners 710 and 730 transmitted from the behavior detectors 720 and 740, and the like. In addition, the control unit 500 transmits the information, which is obtained by matching the emission time and the scanning direction of the laser light for detection LL1′ to each other, to the image data generator 300.

The laser light for detection LL1′ emitted toward the arm 600 is reflected by the surface (superficial layer) of the arm 600 to become reflected laser light LL1″, and the photodiode 222 receives the reflected laser light LL1″. The image data generator 300 detects a time when the photodiode 222 receives the reflected laser light LL1″ and calculates a time difference between a time (the emission time) when the laser light for detection LL1′ is emitted from the detection laser light emitting device 210 and a time when the photodiode 222 receives the reflected laser light LL1″. In addition, the image data generator 300 calculates the distance between the blood vessel display device 100 and the arm 600 in the scanning direction of the laser light for detection LL1′ on the basis of the time difference.

Moreover, the image data generator 300 detects the amount of reflected laser light LL1″ received by the photodiode 222 in addition to calculating the distance as described above. As described above, the laser light for detection LL1′ is near-infrared laser light and has a characteristic of being absorbed by hemoglobin (erythrocyte) contained in blood flowing through the blood vessel 620. Accordingly, if the blood vessel 620 is present in the superficial layer of a part of the arm 600 to which the laser light for detection LL1′ is irradiated, the amount of reflected laser light LL1″ received by the photodiode 222 is reduced. On the contrary, if the blood vessel 620 is not present in the superficial layer of a part of the arm 600 to which the laser light for detection LL1′ is irradiated, the amount of reflected laser light LL1″ received by the photodiode 222 is increased compared with the case where the blood vessel is present. Using such a light amount difference, the image data generator 300 detects whether or not the blood vessel 620 is present in the superficial layer of a part of the arm 600 to which the laser light for detection LL″ is irradiated.

Moreover, in order to determine whether or not the blood vessel 620 is present in a part to which the laser light for detection LL1′ is irradiated, the image data generator 300 may set a threshold value of the amount of reflected laser light LL1″ received by the photodiode 222. In this case, since the image data generator 300 can determine that the blood vessel 620 is present if the amount of light is equal to or smaller than the threshold value and can determine that the blood vessel 620 is not present if the amount of light is larger than the threshold value, it becomes easy to determine whether or not the blood vessel 620 is present. The threshold value can be calculated in advance by experiment or the like.

After emitting the laser light for detection LL1′ as described above, the control unit 500 emits the pulsed laser light for detection LL′ (laser light for detection LL2′, LL3′, LL4′, . . . ) continuously from the detection laser light emitting device 210 at predetermined intervals. In addition, for the laser light for detection LL′, the image data generator 300 detects the distance between the blood vessel display device 100 and the arm 600 in the scanning direction of the laser light for detection LL′ and whether or not the blood vessel 620 is present in a part to which the laser light for detection LL′ is irradiated, similar to the above-described laser light for detection LL1′.

In this way, the image data generator 300 detects the surface shape of the irradiated part 610 of the arm 600 to which the laser light for detection LL′ is irradiated and the arrangement of the blood vessel 620 present in the superficial layer of the irradiated part 610.

Then, the image data generator 300 generates the image data 900D of the image 900 displayed in the irradiation region on the basis of the detected surface shape of the irradiated part 610 and the arrangement of the blood vessel 620. The image 900 is an image for visualizing the blood vessel 620 (which enables the blood vessel 620 to be more easily viewed). The image 900 is not particularly limited if it can visualize the blood vessel 620. For example, an image obtained by irradiating the laser light for display LL to a place other than a part of the irradiated part 610 where the blood vessel 620 is present as shown in FIG. 1 or, on the contrary, an image obtained by irradiating the laser light for display LL only to a part of the irradiated part 610 where the blood vessel 620 is present may be mentioned. Hereinafter, for the sake of convenience, the image 900 obtained by irradiating the laser light for display LL to a place other than a part of the irradiated part 610 where the blood vessel 620 is present as shown in FIG. 1 will be described as representative.

The image data generator 300 can obtain the image data 900D by determining matching regarding whether to irradiate the laser light for display LL for each part to which the laser light for detection LL′ is irradiated. That is, the matching is preferably determined such that the laser light for display LL is not irradiated to a part to which the laser light for detection LL1′ is irradiated when the blood vessel 620 is present in the part and the laser light for display LL is irradiated to a part to which the laser light for detection LL2′ is irradiated when the blood vessel 620 is not present in the part, for example. As a result, it is possible to easily obtain the image data 900D.

The image data 900D generated by the image data generator 300 as described above is transmitted to the control unit 500. In addition, the control unit 500 controls driving of the display unit 400 on the basis of the received image data 900D.

Display Unit 400

The display unit 400 has a function of displaying the image 900, which visualizes the blood vessel 620, on the irradiated part 610 by scanning the irradiated part 610 with the laser light for display LL on the basis of the image data 900D generated by the image data generator 300.

As shown in FIG. 2, the display unit 400 includes a display laser light emitting device 410 which emits the laser light for display LL and a display laser light scanning section 700′ which scans the irradiated part 610 of the arm 600 with the laser light for display LL emitted from the display laser light emitting device 410. By adopting such a configuration, the configuration of the display unit 400 becomes simple.

The display laser light emitting device 410 includes laser light sources 411r, 411g, and 411b of respective colors and collimator lenses 412r, 412g, and 412b and dichroic mirrors 413r, 413g, and 413b provided corresponding to the laser light sources 411r, 411g, and 411b.

The laser light sources 411r, 411g, and 411b of each color emit red, green, and blue laser beams RR, GG, and BB, respectively. The laser beams RR, GG, and BB are emitted in a state modulated corresponding to the driving signal (image data 900D) transmitted from the control unit 500. Then, the laser beams RR, GG, and BB are collimated by the collimator lenses 412r, 412g, and 412b to become narrow beams.

The dichroic mirrors 413r, 413g, and 413b have characteristics of reflecting the red laser beam RR, the green laser beam GG, and the blue laser beam BB, respectively. The laser beams RR, GG, and BB of respective colors are mixed to be emitted as one laser beam for display LL. The laser light for display LL emitted from the display laser light emitting device 410 reaches the display laser light scanning section 700′.

In the present embodiment, since the display laser light emitting device 410 has the laser light sources 411r, 411g, and 411b of respectively colors, any color can be set as a color of the laser light for display LL. Therefore, for example, the color of the laser light for display LL can be changed according to a doctor's liking or the color of a patient's skin. As a result, the convenience of the blood vessel display device 200 is improved.

The display laser light scanning section 700′ scans the irradiated part 610 of the arm 600 in a two-dimensional manner with the laser light for display LL emitted from the display laser light emitting device 410 and depicts the image 900 on the irradiated part 610. As shown in FIG. 2, the detection laser light scanning section 700 of the present embodiment also serves as the display laser light scanning section 700′. As a result, the configuration of the blood vessel display device 100 becomes simple. In addition, since the laser light for display LL and the laser light LL′ for detection scanning the irradiated part 610 using the same light scanning section, the image 900 positioned correctly without deviation can be depict on the irradiated part 610 scanned by the laser light for detection LL′. As a result, a doctor can obtain the blood vessel 620 more reliably.

### Control Unit 500

The control unit 500 controls driving of the detector 200 and also controls driving of the display unit 400 on the basis of the image data 900D generated by the image data generator 300. By providing such a control unit 500, the image 900 can be more reliably displayed on the surface of the arm 600.

Next, control of driving of the display unit 400 by the control unit 500 will be described. First, the control unit 500 drives the first and second optical scanners 710 and 730. Then, on the basis of the behavior information acquired from the behavior detectors 720 and 740 and the image data 900D generated by the image data generator 300, the control unit 500 controls an operation of the display laser light emitting device 410 such that the laser light for display LL is emitted at a predetermined timing.

For example, if the image data 900D is recorded such that the laser light for display LL is not irradiated to a part to which the laser light for detection LL1′ is irradiated, the control unit 500 does not allow the laser light for display LL to be emitted from the display laser light emitting device 410 when the postures of the first and second optical scanners 710 and 730 (movable plates 711a and 731a) match the postures when the laser light for detection LL1′ is emitted. If the image data 900D is recorded such that the laser light for display LL is irradiated to a part to which the laser light for detection LL2′ is irradiated, the control unit 500 allows the laser light for display LL to be emitted from the display laser light emitting device 410 when the postures of the first and second optical scanners 710 and 730 (movable plates 711a and 731a) match the postures when the laser light for detection LL2′ is emitted. When the control unit 500 performs such control over the entire range of the irradiated part 610, the image 900 is depicted on the irradiated part 610 by the display unit 400.

Here, it is preferable to use green laser light as the laser light for display LL which is used for depicting the image 900. Since green is a color easily visible to a human being, a blood vessel can be visualized more reliably. Therefore, a doctor can obtain a blood vessel more reliably. In addition, in the case of using green laser light as the laser light for display LL, the laser light sources 411r and 411b, the collimator lenses 412r and 412b, and the dichroic mirrors 413r and 413b may be removed from the display laser light emitting device 410.

Until now, the blood vessel display device 100 has been described in detail. According to such a blood vessel display device 100, it is possible to display the image 900 (image which visualizes the blood vessel 620), which is clear and has no positional deviation from the actual blood vessel, on the surface of the arm 600. In addition, since laser light is used as display light, the image 900 which is clear and has no blur can be displayed even if the surface of the arm 600 (irradiated part 610) is complexly curved. As a result, a doctor or the like can obtain the blood vessel reliably.

Here, it is preferable that the control unit 500 drives the detector 200 at predetermined intervals and makes the image data generator 300 generate the new image data 900D. Accordingly, even if the position of the arm 600 is displaced with respect to the blood vessel display device 100, the image 900 with no positional deviation from the actual blood vessel 620 can be continuously displayed on the irradiated part 610 because the image 900 follows the displacement. As a result, more reliable and safe medical treatment can be provided. Moreover, although the predetermined interval is not particularly limited, it is preferably equal to or longer than about 0.5 seconds and equal to or shorter than about 1 second.

### Second Embodiment

Next, a blood vessel display device according to a second embodiment of the invention will be described.

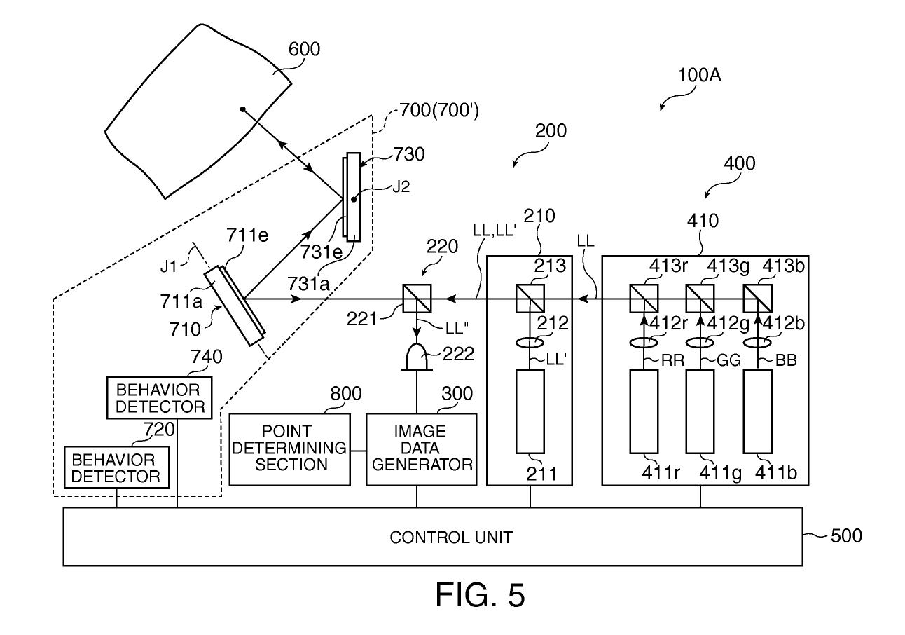

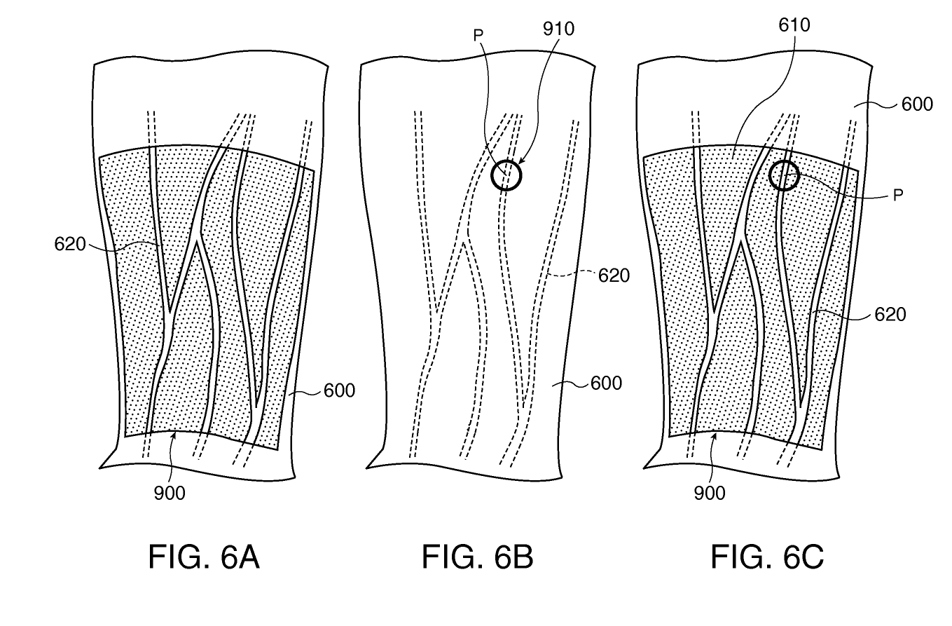

FIG. 5 is a schematic view showing the blood vessel display device according to the second embodiment of the invention. FIGS. 6A to 6C are views showing an example of an image displayed by a display unit shown in FIG. 5.

Hereinafter, a blood vessel display device 100A according to the second embodiment will be described focusing on a point of difference from the above blood vessel display device 100 according to the first embodiment, and explanations of the same subjects will be omitted.

The blood vessel display device 100A according to the second embodiment is almost the same as the blood vessel display device according to the first embodiment except that a point determining section 800 is provided and an image displayed on the irradiated part 610 is different. In addition, the same components as in the first embodiment described above are denoted by the same reference numerals.

As shown in FIG. 5, the blood vessel display device 100A according to the present embodiment has the point determining section 800. The point determining section 800 determines a part (target part P) into which, for example, an injection needle is inserted on the basis of the image data 900D generated by the image data generator 300. The method of determining the target part P is not particularly limited. For example, it may be a part where the blood vessel 620 is relatively thick or may be a part where the blood vessel 620 extends relatively straightly. In addition, determination of the target part P may be automatically performed by the point determining section 800 or may be performed when an operator, such as a doctor, gives a command to the point determining section 800.

The information on the target part P determined by the point determining section 800 as described above is transmitted to the image data generator 300. On the basis of the information transmitted from the point determining section 800, the image data generator 300 generates image data 910D corresponding to an image 910 for displaying the target part P on the irradiated part 610 (for example, an image with a round mark displayed on the target part P).

Moreover, in this case, it is preferable that the color of the laser light for display LL, which is irradiated to the irradiated part 610 in order to display the image 910, is different from the color of the laser light for display LL, which is irradiated to the irradiated part 610 in order to display the image 900. Therefore, a doctor or the like can easily see the target part P.

The image data generator 300 transmits to the control unit 500 the image data 900D and 910D generated as described above. The control unit 500 which has received the image data 900D and 910D performs control of the display unit 400 based on the image data 900D and control of the display unit 400 based on the image data 910D alternately, for example. That is, the control unit 500 controls driving of the display unit 400 so that a state where the image 900 is displayed on the irradiated part 610 as shown in FIG. 6A and a state where the image 910 is displayed on the irradiated part 610 as shown in FIG. 6B are alternately repeated. As a result, an image 920 obtained by overlapping of the images 900 and 910 as shown in FIG. 6C seems to be displayed on the irradiated part 610 to the human eye. Thus, by displaying the target part P, it becomes easier to obtain the blood vessel 620 when giving an injection (when inserting an injection needle beneath the skin). In addition, the mark indicating the target part P may be made to blink, or the size may be temporally changed.

In addition, displaying the images 900 and 910 alternately as in the present embodiment is effective when changing the position of the target part P, for example. That is, when changing the target part P, it is sufficient to just newly generate only the image data 910D of the image 910 (that is, it is not necessary to newly generate the image data 900D). Accordingly, the target part P can be easily changed.

Also in the second embodiment, the same effects as in the first embodiment can be achieved.

While the blood vessel display devices according to the embodiments of the invention have been described with reference to the accompanying drawings, the invention is not limited thereto, and the configuration of each section may be replaced with any configuration with the same function. In addition, adding another structure is also included in the invention. In addition, the invention may be realized by the combination of two or more arbitrary configurations (characteristics) of the embodiments described above.

Moreover, although the case where the display laser light emitting device includes light sources of three colors has been described in the above embodiments, the invention is not limited thereto. For example, the display laser light emitting device may include a light source of only one color.

Moreover, in the above embodiments, the case where the detection laser light scanning section also serves as the display laser light scanning section has been described. However, the invention is not limited thereto, and the detection laser light scanning section and the display laser light scanning section may be separately provided.

In addition, although the case where the detection laser light scanning section has two optical scanners has been described in the above embodiments, the invention is not limited thereto. For example, at least one of the two optical scanners maybe replaced with a galvano mirror. Alternatively, it is also possible to adopt a configuration using one optical scanner, which has a movable plate that can rotate around each of two axes perpendicular to each other, instead of two optical scanners.

Moreover, in the above second embodiment, the case where the target part is a part into which an injection needle is inserted has been described. However, the invention is not limited to this. For example, it may be a target part, such as a guide wire or a catheter.

The entire disclosure of Japanese Application No. 2010-085834, filed Apr. 2, 2010 is expressly incorporated by reference herein.

What is claimed is:

1. A blood vessel display device comprising:
a detection unit that scans body tissue with laser light for detection and detects reflected light from an irradiated part of the body tissue to which the laser light for detection has been irradiated;
an image data generating unit that detects a surface shape of the irradiated part and the arrangement of blood vessels, which are present in a superficial layer of the irradiated part, on the basis of a detection result of the detection unit and generates image data of an image for visualizing the blood vessels displayed on the irradiated part; and
a display unit that displays an image for visualizing the blood vessels on the irradiated part by scanning the irradiated part with laser light for display on the basis of the image data generated by the image data generating unit.

2. The blood vessel display device according to claim 1, further comprising:
a control unit that controls driving of the detection unit and also controls driving of the display unit on the basis of the image data generated by the image data generating unit.

3. The blood vessel display device according to claim 1,
wherein the detection unit includes a detection laser light source which emits the laser light for detection, a detection laser light scanning section which scans the body tissue with the laser light for detection emitted from the detection laser light source, and a light receiving element which receives the reflected light, and
the display unit includes a display laser light source which emits the laser light for display and a display laser light scanning section which scans the irradiated part with the laser light for display emitted from the display laser light source.

4. The blood vessel display device according to claim 3,
wherein each of the detection laser light scanning section and the display laser light scanning section includes an actuator which is provided such that a movable plate including a light reflecting section with light reflectivity is rotatable in at least one direction and which scans the irradiated part with laser light reflected from the light reflecting section due to the rotation.

5. The blood vessel display device according to claim 3,
wherein the detection laser light scanning section also serves as the display laser light scanning section.

6. The blood vessel display device according to claim 1,
wherein the laser light for detection is near-infrared laser light.

7. The blood vessel display device according to claim 1,
wherein the image for visualizing the blood vessels is displayed by the green laser light for display.

8. The blood vessel display device according to claim 1,
wherein the display unit visualizes the blood vessels and displays an image of a target part of the blood vessels.

9. The blood vessel display device according to claim 8,
wherein the target part is a part into which an injection needle is inserted.

10. The blood vessel display device according to claim 1,
wherein the image data generating unit generates the image data at predetermined intervals.
